(19) United States
(12) Patent Application Publication
(10) Pub. No.: US 2016/0022755 A1
(43) Pub. Date: Jan. 28, 2016

(54) HERBAL GEL FROM CHESTNUT SEED COAT FOR PELVIC FLOOR MUSCLE RELAXATION IN WOMEN AND A METHOD OF SYNTHESIZING THE SAME

(71) Applicant: Fariba Sepahvand, Khoram Abad (IR)

(72) Inventor: Fariba Sepahvand, Khoram Abad (IR)

(21) Appl. No.: 14/873,203

(22) Filed: Oct. 2, 2015

(51) Int. Cl. A61K 36/49; A61K 9/06

(52) U.S. Cl. CPC A61K 36/49; A61K 9/06

(57) ABSTRACT

The embodiments herein disclose a method for extracting the herbal extract from acorn or oak nut of Quercus brantii for synthesizing a herbal gel for targeting a vaginal relaxation syndrome (VRS) and a pelvic organ prolapsed (POP). The acorn or oak nut from Quercus brantii trees is collected. The wooden shell is separated and the seed coat is separated. The seed coat is milled into a powder. The herbal extract from the seed coat powder is extracted by the maceration method. The solvent used is methanol. The herbal extract is mixed with methyl paraben (0.2% w/w), acetic acid, de-ionized water and glycerine to obtain a gel. The herbal gel is applied to the women for three days. After application of the herbal gel the women experience more sexual satisfaction, an orgasm, a vaginal stenosis and a slippery vagina during the sexual intercourse. The women experience relief in VRS and POP.

### SPONSORSHIP STATEMENT

This application is financially sponsored for international filing by the IRANIAN NATIONAL SCIENCE FOUNDATION (INSF).

### BACKGROUND

1. Technical field

The embodiments herein generally relates to the field of herbal composition. The embodiments herein particularly relate to synthesis of herbal gel from chestnut. The embodiment herein more particularly relate to synthesis of herbal gel from chestnut placenta and application of herbal gel for muscle relaxation. The embodiments herein also relate to the application of herbal gel for pelvic muscle relaxation in women.

2. Description of the Related Art

Quercus brantii or the Persian oak is a species of oak native to Western Asia including Iran, Iraq, Syria and Turkey. Quercus brantii is an important tree species of the Zagros mountain range in Iran. In Iran the seeds of Quercus brantii are used in traditional medicines for inflammatory and gastric ulcers. Other useful products derived from oaks include fuel wood, charcoal and timber hardwood.

Quercus brantii or the Persian oak nut or seed is used in traditional medicine. The branches and leaves are hairy so the leaves are gray-green. They are leathery and oval shaped. Iranian oak tree produces both male flowers and small female flowers which are on the same tree, and they bear fruit in a height between 3-4 meters. The fruit is an oval nut borne in a cup like structure and weight about 15-20 gram.

Fruit of Iranian chestnut comprises of two parts known as cup and capsule. The capsule is also divided to three segments. the three segments are shell or husk, placenta or hull and pith. The shell or husk of seed surrounds the capsule and holds 7% of the fruit weight. The placenta or hull/husk of seed comprises of tannins. The placenta or hull of seed is a thin hull that covers pith (second hull of seed). The placenta or hull of seed region comprises 3% of dried fruit and 30 gram placenta is obtained from 1 Kilogram of dried chestnut,. The chestnut's pith is the hull of chestnut seed. The pith is soft until the pith is not dried. As the chestnuts get dried due to internal water evaporation, the weight of the pith decreases. 40% of the unripe chestnut fruit comprises water and salts. The chestnuts are divided to evergreen or deciduous trees or shrubs with alternate and simple leaves. The chestnut fruit is like a circle enclosed in a cup called “cupule”. Shikimic acid, methyl salicylate, terpenoid compounds, and especially tannins are found in these plants. Chestnut hull/husk is a good antidote for alkaloids and metals poisonings.

Several chestnut species have found various applications and usage. The Fagus Sylvatica chestnut fruit is used for oil production. The hulls of different species of chestnut consist of a lot of tannins and are used for extraction of tannin material. Quercus stenophylla is used for producing yellow colour. The tannic acid is also extracted from the leaves of Quercus infectoria and Quercus brantii seed hull. The chestnut species are used in traditional medicines. Quercus stenophylla juice is used for accelerating the excretion of kidney and urinary tract stones. The polyphenolic tannins are used in medical and pharmacy as an astringent, anti-diarrheal, anti-bleeding and skin healer. The tannins are also used in jewellery industry and tannery. Quercus infectoria extracts are also used for synthesis of sweetener and laxative.

The hull of aforementioned species is called as placenta. The hull is also known as husk or placenta. In botany, the husk or hull or placenta is the outer shell or coating of seed. The hull or husk includes the protective outer covering of a seed, fruit or vegetable. The hull or placenta of chestnut is rich in tannins. The chestnut fruit and hull have tannins with astringent and disinfectant effects.

According to previous studies, tannins are one of the active components in hull and fruit. The tannins have effect on diarrheal treatment. The tannins are used effectively on the diarrheal treatment because of water absorption and proteins sediment. The previous studies indicate that methanolic juice of fruit (Quercus Lusitanica) has a noticeable inhibitory effect on bone fever Type II virus replication. The inner hull of chestnut's fruit, placenta, has health benefits similar to that of the fruit. The study on herbal mixture of chestnut's inner hull and lemon balm's effect on controlling minor aphthous ulcers of oral mucosa shows that the mixture is significantly useful for treating the illness. In a study on Quercus aucheri, it has been shown that chestnut hull is used as an anti-styptic astringent and blood coagulant. The chestnut herbal tea is used for severe or acute diarrhoea, inflammation, and edema. The chestnut extracts are also used for burns and wounds. Because of the different medicinal properties of tannins such as prevention the growth of pathogens, anti-bleeding, and being astringent, the tannin powder from hulls are used for skin wound healing in rats. In another study on Quercus infectoria for wound healing properties an accelerating wound healing in rats by ethanol juice of plant hull is reported.

The tannins derived from the chestnut hull have an impact on smooth muscles of the gastrointestinal tract that cause diarrhea to stop.

The Vaginal relaxation syndrome (VRS) is defined as laxity of the vaginal wall. The VRS results in a loss of friction and sexual satisfaction for both a woman and her partner. The Vaginal atrophy refers to the thinning of the wall of the vagina. The thinning of the vaginal wall is found post menopause in women and is associated with dryness. The most common symptoms of VRS are the vagina is loose when having intercourse, less or no sensitivity when having intercourse (little or no friction during intercourse), feeling less sensitive in intercourse since vaginal childbirth; symptoms are of vaginal relaxation syndrome.

The cause of the vaginal relaxation syndrome (VRS) is a result of vaginal trauma. The number one cause is vaginal childbirth. The more vaginal delivers a woman undergoes, the greater the trauma and the greater the risk of developing vaginal relaxation syndrome. Other causes of vaginal injury include hysterectomy and pelvic surgery.

Pelvic organ prolapse (POP) is another condition in women. POP is the herniation of the pelvic organs to or beyond the vaginal walls and it is a common condition. Many women with POP condition experience symptoms that impact daily activities, sexual function and exercise. The presence of POP has a detrimental impact on body image and sexuality. Most women are embarrassed by such a problem and do not report it to their doctor. Women with POP often complain of vaginal or pelvic pressure, sensation of a vaginal bulge or something falling out of the vagina. Women with POP often complain of vaginal or pelvic pressure, sensation of a vaginal bulge or something falling out of the vagina. Women also experience associated symptoms including urinary defecatory or sexual dysfunction.

Symptoms such as low back or pelvic pain have often attributed to POP. Other symptoms include stress, urinary incontinence, increased difficult in voiding, obstructed voiding, slow urine stream, the need to change position or manually reduce the prolapsed to urinate a sensation of incomplete emptying and in rare cases complete urinary retention. Overactive bladder symptoms (urgency, urge for urinary incontinence, frequency) are also common. In addition some women with POP experience enuresis or incontinence with sexual intercourse.

The most common bowel symptom associated with prolapsed is constipation. Other defecator symptoms include fecal urgency, fecal incontinence and obstructive symptoms including incomplete emptying straining or the need to apply digital pressure to the vagina or perineum (splint) to completely evacuator. Some women report fecal incontinence during sexual intercourse.

Different treatments are reported for curing prolapsed pelvic organs are surgical or non surgical. Non surgical treatment options include the use of a vaginal pessary (a plastic or rubber ring inserted into vagina to provide surgical support), kegel exercises, physiotherapy and hormonal therapy. Anterior and posterior colporrhaphy are surgical options for pelvic organ laplace.

The success rate of surgery is 85% and its failure rate is about 15%. Hence most of the patients choose surgery. The national institute of health in U.S. reports 400000 colporrhaphy a year. In earlier studies, it is reported that sexual function and sexual satisfaction is increased after surgery.

It is also reported that sexual satisfaction decreases after 3-6 months post-surgery.

The treatment for women with pelvic organ prolapse (POP) and vaginal relaxation syndrome (VRS) is based on the individual patient's symptom and the impact on their quality of life. The women with prolapsed symptomatic can be managed expectantly or treated with conservative or surgical therapy. Both conservative and surgical treatment options should be offered. “Expectant management” (meaning do nothing) is a variable option for women who can tolerate their symptoms and prefer to avoid treatment. “Vaginal pessary” is the mainstay of non-surgical treatment. Pessaries are silicone devices in a variety of shapes and sizes which support the pelvic organ. They are used for pelvic floor muscle exercise, estrogen therapy and surgical treatment.

The non surgical method or non invasive method for targeting or treating vaginal relaxation syndrome (VRS) and pelvic organ prolapse (POP) includes kegel exercise for tightening up relaxed muscles in the genital area or pelvic floor and to a lesser extent in the vaginal wall. Exercises may or may not give relief Further it may take more time to show improvement. Hormonal creams, sprays and pharmacological approaches are also available. However the non invasive techniques are limited in effectiveness and give temporary relief, requiring continuous pharmacotherapy. The hormonal treatment creates the metabolic imbalance and disorders. Further continuous usage drugs or pharmacological composition have many side effects.

The surgical procedure involves incision and rearrangement of vaginal and associated tissues. The surgery must be balanced against the much greater risk involved in surgery of extremely sensitive vaginal tissues.

The astringent effects of tannins from chestnut's hull have impact on smooth muscles of the gastrointestinal tract and thus stop the diarrhea. The same property can treat women who are suffering from genital tract smooth muscle relaxation.

Hence there is a need for an herbal composition comprising tannins for targeting or treating vaginal relaxation syndrome (VRS) and pelvic organ prolapse (POP). Also there is a need for a simple method for extracting the tannins from chestnut.

The above mentioned shortcomings, disadvantages and problems are addressed herein and which will be understood by reading and studying the following specification.

### OBJECTIVES OF THE EMBODIMENTS

The primary objective of the embodiment herein is to provide a herbal composition for targeting vaginal relaxation syndrome (VRS) and pelvic organ prolapsed (POP) synthesized from Quercus brantii seed.

Another object of the embodiment herein is to provide a simple method for synthesis of herbal gel from Quercus brantii seed coat/hull.

Yet another object of the embodiment herein is to provide a herbal gel which is easy to apply in vagina.

Yet another object of the embodiment herein is to provide a herbal gel which is effective at a dosage of 20 gram.

Yet another object of the embodiment herein is to provide a herbal gel which increases the sexual satisfaction, orgasm, vaginal stenosis and slippery vagina during intercourse.

Yet another object of the embodiment herein is to provide a herbal gel from Quercus brantii hull which prevents pathogen growth, anti-hemorrhagic and anti-bleeding.

Yet another object of the embodiment herein is to provide a herbal gel from Quercus brantii seed coat/hull which has no side effects.

Yet another object of the embodiment herein is to provide a herbal gel from Quercus brantii seed coat/hull which has a low cost.

These objects and the other advantages of the embodiments herein will become readily apparent from the following detailed description taken in conjunction with the accompanying drawings.

### SUMMARY

The various embodiments herein provide an herbal composition for targeting vaginal relaxation syndrome (VRS) and pelvic organ prolapsed (POP). Further a simple method is used for synthesis of herbal gel from Quercus brantii seed coat/hull.

According one embodiment herein, composition for targeting a vaginal relaxation syndrome (VRS) and a pelvic organ prolapsed (POP) is provided. The composition comprises a herbal extract powder of Quercus brantii seed coat (hull), methyl paraben 0.2% w/w) an acetic acid, de-ionized water and glycerine.

According to one embodiment herein, the herbal extract powder of Quercus brantii seed coat (hull) is present in the composition at a preset weight % range. The preset weight ranges are 1.5% w/w, 2% w/w and 2.5% w/w.

According to one embodiment herein, the methyl paraben is present in the composition at a preset weight %. The preset weight % of the methyl paraben is 0.2% w/w.

According to one embodiment herein, the herbal gel is administered to women (candidates/participants/volunteers) with a vaginal relaxation syndrome (VRS) and a pelvic organ prolapsed (POP).

According to one embodiment herein, a method is provided for synthesizing a herbal gel comprising the herbal extract from acorn or oak nut of Quercus brantii for targeting a vaginal relaxation syndrome (VRS) and a pelvic organ prolapsed (POP). The method comprises the following the steps. A herbal extract powder of Quercus brantii seed coat (hull), methyl paraben (0.2% w/w), acetic acid, de-ionized water and glycerine are mixed to obtain the herbal extract gel of Quercus brantii seed coat (hull) extract.

According to one embodiment herein, the herbal extract powder of Quercus brantii seed coat (hull) is mixed in a preset weight % range. The preset weight % ranges are 1.5% w/w, 2% w/w and 2.5% w/w.

According to one embodiment herein, the methyl paraben is mixed in a preset weight and wherein the preset weight % of the methyl paraben is 0.2% w/w.

According to one embodiment herein the herbal gel is administered to women (candidates/participants/volunteers).

According to one embodiment herein, the women (candidates/patients/participants) are administered with plurality of dosages of herbal gel, and wherein the plurality of dosage of herbal gel is 10 gram of herbal gel, or 15 gram of herbal gel and 20 gram of herbal gel. The herbal gel is administered for days.

According embodiment to on herein, the 10 gram of herbal gel comprises of 1.5% w/w Quercus brantii hull/seed coat extract. The 15 gram of herbal gel comprises of 2% w/w Quercus brantii hull/seed coat extract. The 20 gram of herbal gel comprises of 2.5% w/w Quercus brantii hull/seed coat extract.

According to one embodiment herein, the women (patients/volunteers/candidates) experience enhanced sexual satisfaction, orgasm, vaginal stenosis and slippery vagina during the sexual intercourse, after the application of the herbal gel with Quercus brantii hull/seed coat extract in a concentration of 2.5% w/w.

According to one embodiment herein, 20 gram of the herbal gel with Quercus brantii hull/seed coat extract increases an effect on the women (patients/volunteers/candidates) with vaginal relaxation syndrome (VRS) and pelvic organ prolapsed (POP).

According to one embodiment herein, a method is provided for extracting a herbal extract from acorn or oak nut of Quercus brantii for synthesizing a herbal gel for targeting a vaginal relaxation syndrome (VRS) and pelvic organ prolapsed (POP). The method comprises the following steps. The acorn or oak nut from Quercus brantii trees are collected. The acorn or oak nuts are dried at a room temperature. The wooden shells are separated from the acorn or oak nut seed. The seed coats present below the wooden shell are separated from each of the acorn or oak nut The seed coat is milled into a powder. The seed coat powder is mixed with a solvent in a capped container to get a mixture. The mixture is incubated at a room temperature for a predetermined period of time. The predetermined time period is 4-72 hours. The mixture is agitated at a regular interval of time for dissolving tannins. The mixture is strained after 48 hours. The solids of the mixture are pressed to extract the solvent and the solvent is methanol extract. The solvent is cleaned by plurality of methods. The plurality of methods includes decanting or filtering. The herbal extract filtrate is obtained by evaporating the methanol extract. The herbal extract is cooled to obtain a powder at a room temperature. The herbal extract is powdered into a powder room temperature. The herbal extract powder is stored in a refrigerator. The solvent is methanol. The concentration of the methanol is 80% v/v.

According to one embodiment herein, Iranian acorns of Quercus brantii consist of cupule and pericarp. The seed has three main parts. The external shell is cupule (base of an acorn) and comprises 7% of acorns dry weight. The second layer is seed coat which is a very thin layer around the cotyledons and comprises 3% of acorn dry weight. Hence 30g seed coat is obtained from each 1 kg dry acorn. The last part is cotyledons. The kernel is soft when the acorn is not dry. But as the time passes the kernel lose weight because of the vaporization of its water. Water and minerals comprises 40% weight of raw acorn. This genus includes trees or shrubs which are deciduous or evergreen. The fruit is a nut called acorn, borne in a cup like structure known as cupule. These plants contain shikimic acid, methyl salicilate, terpenoids and especially tannin. Acorn hull is a good antidote for alkaloid and metal poisoning. Tannins (polyphenolic compounds) are used as styptic, anti diarrhea, and anti-bleeding in medicine and pharmacy. It is styptic and disinfectant.

According to one embodiment herein, in autumn the acorn or oak nut from Quercus brantii is collected from Dorood region in Khoram Abad and transferred to the laboratory. The oak nuts are dried at room temperature and the wooden shells are separated. The inner layer under the wooden shell is called as seed coat. The seed coats are taken out from each oak nut seed. The seed coats are milled into a powder. For the extractions of tannins maceration process is used. The seed coat powder is placed with the solvent in a capped container. The solvent is 80% methanol. The container is incubated at room temperature for a period of 48-72 hours with frequent or regular agitation.

The agitation helps in dissolving of the tannins. After 48 hours the methanol and seed coat powder mixture is strained and the solid material in mixture is pressed to extract the solvent. The filtrate is clarified by filtration or decantation. The filtrate obtained is evaporated to obtain the methanol extract. The evaporated herbal extract is cooled in a desiccator. The extract is made into powder in room temperature. The powder is stored in the fridge.

According to one embodiment herein, the main component of the extract is tannic quercus acid and gallic acid. The herbal gel is synthesized by mixing herbal extract, methyl paraben (0.2% w/w), acetic acid, de-ionized water and glycerin.

According to one embodiment herein, this embodiment here target treatment for 120 married women with different pelvic organ prolapse who are candidate for colporrhaphy surgery. The herbal gel is used for first time for targeting the pelvic organ prolapsed in women.

According to one embodiment herein, first a study on women suffering from different kind of pelvic organ prolapse is conducted. The women are the patients who attended to the clinic of Asali Educational Hospital. The women are selected in order to pursue, diagnose and remedy their genital organs disease. The participants or volunteer women are selected who are married, who have husband and children, educated in school or higher level education, women who didn't experienced divorce, pelvic and breast surgery, narcotic or anti-depression or psychotherapy drugs (both, wife and husband), chronic disease like high blood pressure or diabetes, sexual problems of their husband (premature ejaculation, erectile dysfunction), urinary tract infection (UTI) and genital infection, or any mental tension (e.g., refractory disease of family member, changing the place they live, loss of a dear one, retirement of the woman or her husband) in the last 6 months before the experiment.

According to one embodiment herein, the aim of the embodiment herein is the comparison of the effect of Quercus Oak herbal gel in contraction of vaginal muscles in women with vaginal relaxation or vaginal relaxation syndrome (VRS) and pelvic organ prolapsed (POP). 120 women with relaxation of vaginal muscles or vaginal relaxation syndrome (VRS) and pelvic organ prolapsed (POP), candidates with colporaphy surgery since 22 Aug. 2012 to 21 May 2015 and candidates who are married. The candidates must not have history of chronic disease (such as diabetes, blood pressure, cardiac diseases). The husband of the candidates must not have sexual problems (such as sexual weakness, premature ejaculation) and genito urinary infection. The sampling of the candidates is done according to number of references in the hospital. The aim/objective of the study is explained in a simple manner and the candidates are made aware to participate. All the women (candidates/patients/participants) are classified for the degree of pelvic organ prolapsed according to pelvic organ prolapsed quantification system (POP-Q) standard. The women (candidates/patients/participants) are subjected to lithotomy checkup for analyzing the degree of the vaginal relaxation syndrome (VRS) and pelvic organ prolapsed (POP).

Before the prescribing the herbal gel synthesized from the Quercus brantii hull the candidates are subjected to the questionnaire to get the details. The questionnaire number 1 comprises the information of disease demographic in candidates; questionnaire number 2 comprises the questionnaire of sexual satisfaction PISQ.

According to one embodiment herein, all the women are examined in lithotomic position and graded according to pop-Q. After examination the women (candidates/patients/participants) are divided into four groups, fifteen women in each, considering the age of the women in each group (the women candidates are of same age range). One of the groups is control group. Different dose of medicine is prescribed to each group. In order to blind the experiment another person does the allocation part so the researcher isn't aware of the allocation. The patients aren't aware about the dosage and placebo. After explaining the purposes of the study to the patients/participants and receiving testimonial, the patients/participants fill questionnaire (1) (patients/participants demographic information) and the questionnaire about sexual satisfaction. Then the women (candidates/patients/participants)are prescribed with different dose of medicine (10 gram herbal gel, 15gram herbal gel and 20gram herbal gel, comprising 1.5% w/w Quercus brantii hull extract, 2% w/w Quercus brantii hull extract and 2.5% w/w Quercus brantii hull extract respectively). The control group is prescribed the same gel without acorn extract (the basis of gel includes methyl paraffin 0.2%, acetic acid, de-ionized water and glycerin). Different doses of this extract are prescribed for a period of 3 days, after menstruation and before having sexual intercourse. After application of the last dose the patient/participants are prescribed to have sex. One day after application of the herbal gel effectiveness of the herbal gel and adverse effect of herbal gel are recorded.

After application of the herbal gel the candidates are allowed for sexual intercourse. The day after having sexual intercourse all the groups are studied for side effects and fill questionnaire number 2 (sexual satisfaction questionnaire PISQ). The marks are classified in 5 levels: completely satisfied (81-100), partly satisfied (61-80), no idea (41-60), partly unsatisfied (21-40), and completely unsatisfied (0-20). The credibility of the tools is counted through credibility of content, and the questionnaire reliability is counted through Chronbach's alpha test (Chronbach sexual satisfaction questionnaire 89%). In order to determine the reliability of physical examination and access the degree of pelvic organ prolapse, correlation coefficients (spearman r=96%) is used. The person who peruses the participants isn't aware about the way of allocation. The data in information forms are classified and extracted after using medicine.

According to one embodiment herein, after the application of the herbal gel with Quercus brantii hull extract herbal gel (acorn seed coat vaginal gel) with a concentration of 2.5% w/w, the women (patients/candidates) experience more sexual satisfaction, orgasm, vaginal stenosis and a slippery vagina during sexual intercourse when compared to the other groups.

According to one embodiment herein, the sexual satisfaction, orgasm, vaginal stenosis and a slippery vagina during intercourse are found to be less when the Quercus brantii hull extract herbal gel (acorn seed coat vaginal gel) is applied at a dose of in 10 gram and 15 gram dose. After using the herbal gel at a dosage of 20 gram the sexual satisfaction, orgasm, vaginal stenosis and a slippery vagina during intercourse increased.

According to one embodiment herein, 20 g of the herbal gel with Quercus brantii hull extract herbal gel (acorn seed coat vaginal gel) has more effect on the vaginal relaxation syndrome (VRS) and pelvic organ prolapsed (POP).

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart indicating a method for extracting the herbal extract from acorn or oak nut of Quercus brantii, according to one embodiment herein.

FIG. 2 illustrates a flowchart indicating a method for synthesizing the herbal gel comprising herbal extract from acorn or oak nut of Quercus brantii, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide an herbal composition for targeting vaginal relaxation syndrome (VRS) and pelvic organ prolapsed (POP). Further a simple method is used for synthesis of herbal gel from Quercus brantii seed coat/hull.

According to one embodiment herein, a composition for targeting a vaginal relaxation syndrome (VRS) and a pelvic organ prolapsed (POP) is provided. The composition comprises a herbal extract powder of Quercus brantii seed coat (hull), methyl paraben (0.2%), an acetic acid, de-ionized water and glycerine.

According to one embodiment herein, the herbal extract powder of Quercus brantii coat (hull) is present in the composition at a preset weight % range. The preset weight % ranges are 1.5% w/w, 2% w/w and 2.5% w/w.

According to one embodiment herein, the methyl paraben is present in the composition at a preset weight %. The preset weight % of the methyl paraben is 0.2% w/w.

According to one embodiment herein, the herbal gel is administered to women (candidates/participants/volunteers) with a vaginal relaxation syndrome (VRS) and a pelvic organ prolapsed (POP).

According to one embodiment herein, a method is provided for synthesizing a herbal gel comprising the herbal extract from acorn or oak nut of Quercus brantii for targeting a vaginal relaxation syndrome (VRS) and a pelvic organ prolapsed (POP). The method comprises the following the steps. A herbal extract powder of Quercus brantii seed coat (hull) methyl paraben (0.2% w/w), acetic acid, de-ionized water and glycerine are mixed to obtain the herbal extract gel of Quercus brantii seed coat (hull) extract.

According to one embodiment herein, the herbal extract powder of Quercus brantii seed coat (hull) is mixed in a preset weight % range. The preset weight % ranges are 1.5% w/w, 2% w/w and 2.5% w/w.

According to one embodiment herein, the methyl paraben is mixed in a preset weight %, and wherein the preset weigh of methyl paraben is 0.2% w/w.

According to one embodiment herein, the herbal gel is administered to women (candidates/participants/volunteers).

According to one embodiment herein, the women (candidates/patients/participants) are administered with plurality of dosages of herbal gel and wherein the plurality of dosage of herbal gel is 10 gram of herbal gel, or 15 gram of herbal gel and 20 gram of herbal gel. The herbal gel is administered for 3 days.

According to one embodiment herein, the 10 gram of herbal gel comprises of 1.5% w/w Quercus brantii hull/seed coat extract. The 15 gram of herbal gel comprises of 2% w/w Quercus brantii hull/seed coat extract. The 20 gram of herbal gel comprises of 2.5% w/w Quercus brantii hull/seed coat extract.

According to one embodiment herein, the women (patients/volunteers/candidates) experience enhanced sexual satisfaction, orgasm, vaginal stenosis and slippery vagina during the sexual intercourse, after the application of the herbal gel with Quercus brantii hull/seed coat extract in a concentration of 2.5% w/w.

According to one embodiment herein, 20 gram of the herbal gel with Quercus brantii hull/seed coat extract increases an effect on the women (patients/volunteers/candidates) with vaginal relaxation syndrome (VRS) and pelvic organ prolapsed (POP).

According to one embodiment herein, a method is provided for extracting a herbal extract from acorn or oak nut of Quercus brantii for synthesizing a herbal gel for targeting a vaginal relaxation syndrome (VRS) and a pelvic organ prolapsed (POP). The method comprises the following steps. The acorn or oak nut from Quercus brantii trees are collected. The acorn or oak nuts are dried at a room temperature. The wooden shells are separated from the acorn or oak nut seed. The seed coats present below the wooden shell are separated from each of the acorn or oak nut. The seed coat is milled into a powder. The seed coat powder is mixed with a solvent in a capped container to get a mixture. The mixture is incubated at a room temperature for a predetermined period of time. The predetermined time period is 4-72 hours. The mixture is agitated at a regular interval of time for dissolving tannins. The mixture is strained after 48 hours. The solids of the mixture are pressed to extract the solvent and the solvent is methanol extract. The solvent is cleaned by plurality of methods. The plurality of methods includes decanting or filtering. The herbal extract filtrate is obtained by evaporating the methanol extract. The herbal extract is cooled to obtain a powder at a room temperature. The herbal extract is powdered into a powder at a room temperature. The herbal extract powder is stored in a refrigerator. The solvent is methanol. The concentration of the methanol is 80% v/v.

According to one embodiment herein, Iranian acorns of Quercus brantii consist of cupule and pericarp. The seed has three main parts. The external shell is cupule (base of an acorn) and comprises 7% of acorns dry weight. The second layer is seed coat which is a very thin layer around the cotyledons and comprises 3% of acorn dry weight. Hence 30 g seed coat is obtained from each 1 kg dry acorn. The last part is cotyledons. The kernel is soft when the acorn is not dry. But as the time passes the kernel lose weight because of the vaporization of its water. Water and minerals comprises 40% weight of raw acorn. This genus includes trees or shrubs which are deciduous or evergreen. The fruit is a nut called acorn, borne in a cup like structure known as cupule. These plants contain shikimic acid, methyl salicilate, terpenoids and especially tannin. Acorn hull is a good antidote for alkaloid and metal poisoning. Tannins (polyphenolic compounds) are used as styptic, anti diarrhea, and anti-bleeding in medicine and pharmacy. It is styptic and disinfectant.

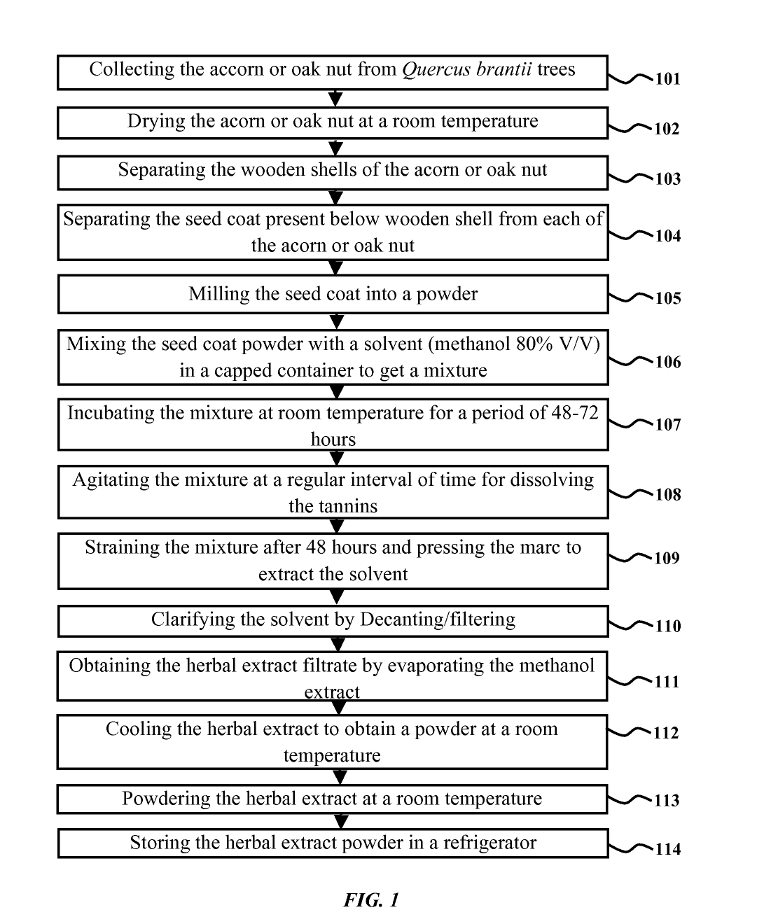

FIG. 1 illustrates a flowchart indicating a method for extracting the herbal extract from acorn or oak nut of Quercus brantii, according to one embodiment herein. The first step is collecting the acorn or oak nut from Quercus brantii trees (101). After collecting the acorn or oak nut, the acorn or oak nut are dried at a room temperature (102). After drying the oak nut or acorn, the wooden shells of the acorn or oak nut are separated (103). The seed coat present below wooden shell is separated from each of the acorn or oak nut (104). The seed coat is milled into a powder (105). The seed coat powder is mixed with a solvent (methanol 80% V/V) in a capped container to get a mixture (106). The mixture is incubated at room temperature for a period of 48-72 hours (107). The mixture is agitated at a regular interval of time for dissolving the tannins (108). The mixture is strained after 48 hours and pressing the marc to extract the solvent (109). The solvent is clarified by decanting/filtering (110). The herbal extract filtrate is obtained by evaporating the methanol extract (111). The herbal extract is cooled to obtain a powder at a room temperature (112). The herbal extract is powdered at a room temperature (113). The herbal extract powder is stored in a refrigerator (114).

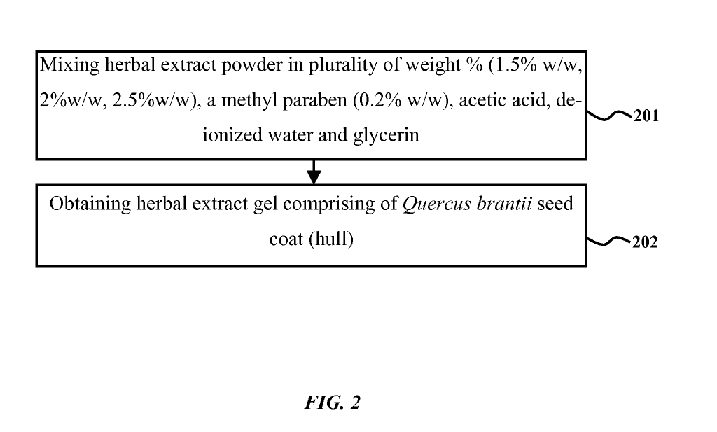

FIG. 2 illustrates a flowchart indicating a method for synthesizing the herbal gel comprising herbal extract from acorn or oak nut of Quercus brantii, according to one embodiment herein. The herbal extract powder is mixed in plurality of weight % (1.5% w/w, 2% w/w, 2.5% w/w), a methyl paraben (0.2% w/w), acetic acid, de-ionized water and glycerin (201). The herbal extract gel is obtained comprising of Quercus brantii seed coat (hull) (202).

According to one embodiment herein, in autumn the acorn or oak nut from Quercus brantii is collected from Dorood region in Khoram Abad and transferred to the laboratory. The oak nuts are dried at room temperature and the wooden shells are separated. The inner layer under the wooden shell is called as seed coat. The seed coats are taken out from each oak nut seed. The seed coats are milled into a powder. For the extractions of tannins maceration process is used. The seed coat powder is mixed with the solvent in a capped container. The solvent is 80% methanol. The container is incubated at room temperature for a period of 48-72 hours with frequent or regular agitation. The agitation helps in dissolving of the tannins. After 48 hours the methanol and seed coat powder mixture is strained and the marc (the solid material in mixture) is pressed to extract the solvent. The filtrate is clarified by filtration or decantation. The herbal extract filtrate obtained is evaporated to obtain the methanol extract. The evaporated herbal extract is cooled in a desiccator. The extract is made into powder in room temperature. The powder is stored in the fridge.

According to one embodiment herein, the main component of the extract is tannic quercus acid and gallic acid. The herbal gel is synthesized by mixing herbal extract, methyl paraben (0.2% w/w), acetic acid, de-ionized water and glycerin.

According to one embodiment herein, this embodiment here target treatment for 120 married women with different pelvic organ prolapse who are candidate for colporrhaphy surgery. The herbal gel is used for first time for targeting the pelvic organ prolapsed in women.

According to one embodiment herein, first a study on women suffering from different kind of pelvic organ prolapse is conducted. The women are the patients who attended to the clinic of Asali Educational Hospital. The women are selected in order to pursue, diagnose and remedy their genital organs disease. The patients/participants or volunteer women are selected who are married, who have husband and children, educated in school or higher level education, women who didn't experienced divorce, pelvic and breast surgery, narcotic or anti-depression or psychotherapy drugs (both, wife and husband), chronic disease like high blood pressure or diabetes, sexual problems of their husband (premature ejaculation, erectile dysfunction),urinary tract infection (UTI) and genital infection, or any mental tension (e.g., refractory disease of family member, changing the place they live, loss of a dear one, retirement of the woman or her husband) in the last 6 months before the experiment.

According to one embodiment herein, the aim of the embodiment herein is the comparison of the effect of Quercus Oak herbal gel in contraction of vaginal muscles in women with vaginal relaxation or vaginal relaxation syndrome (VRS) and pelvic organ prolapsed (POP). 120 women (candidates/patients/participants) with relaxation of vaginal muscles or vaginal relaxation syndrome (VRS) and pelvic organ prolapsed (POP), candidates/patients/participants with colporaphy surgery since 22 Aug. 2012 to 21 May 2015 and candidates/patients/participants who are married. The candidates/patients/participants must not have history of chronic disease (such as diabetes, blood pressure, cardiac diseases). The husband of the candidates/patients/participants must not have sexual problems (such as sexual weakness, premature ejaculation) and genito urinary infection. The sampling of the candidates/patients/participants is done according to number of references in the hospital. The aim/objective of the embodiments is explained in a simple manner and the candidates are made aware to participate. All the women (candidates/patients/participants) candidates are classified for the degree of pelvic organ prolapsed according to pelvic organ prolapsed quantification system (POP-Q) standard. The women (candidates/patients/participants) are subjected to lithotomy checkup for analyzing the degree of the vaginal relaxation syndrome (VRS) and pelvic organ prolapsed (POP).

Before prescribing the herbal gel synthesized from the Quercus brantii hull the women (candidates/patients/participants) are subjected to the questionnaire to get the details. The questionnaire number-1 comprises the information of disease demographic in candidates; questionnaire number-2 comprises the questionnaire of sexual satisfaction PISQ.

According to one embodiment herein, all the women (candidates/patients/participants) are examined in lithotomic position and graded according to POP-Q. After examination the women (candidates/patients/participants) are divided into four groups, fifteen women (candidates/patients/participants) in each, considering the age of the women in each group (the women candidates are of same age range).One of the groups is control group. Different dose of medicine is prescribed to each group. In order to blind the experiment another person does the allocation part so the researcher isn't aware of allocation. The women (candidates/patients/participants) are not aware about the dosage and placebo. After explaining the purposes of the study for the patients and receiving testimonial, the participants fill questionnaire (1) (patients demographic information) and the questionnaire about sexual satisfaction. Then the women (candidates/patients/participants) are prescribed with different dose of medicine (10 gram herbal gel, 15 gram herbal gel and 20 gram herbal gel, comprising 1.5% w/w Quercus brantii hull extract, 2% w/w Quercus brantii hull extract and 2.5% w/w Quercus brantii hull extract respectively). The control group is prescribed the same gel without acorn extract (the basis of gel includes methyl paraffin 0.2%, acetic acid, de-ionized water and glycerin). Different doses of this extract are prescribed for a period of 3 days, after menstruation and before having sexual intercourse. After using the last dose the (candidates/patients/participants) are prescribed to have sex. One day after application of the herbal gel effectiveness of the herbal gel and adverse effect of herbal gel are recorded.

After application of the herbal gel, the women (candidates/patients/participants) are allowed for sexual intercourse. The day after having sexual intercourse all the groups are studied for side effects and fill questionnaire number 2 (sexual satisfaction questionnaire PISQ). The marks are classified in 5 levels: completely satisfied (81-100), partly satisfied (61-80), no idea (41-60), partly unsatisfied (21-40), and completely unsatisfied (0-20). The credibility of the tools is counted through credibility of content, and the questionnaire reliability is counted through Chronbach's alpha test (Chronbach sexual satisfaction questionnaire 89%). In order to determine the reliability of physical examination and access the degree of pelvic organ prolapse, correlation coefficients (spearman r=96%) is used. The person who peruses the participants isn't aware about the way of allocation. The data in information forms are classified and extracted after using medicine.

According to one embodiment herein, after the application of the herbal gel with Quercus brantii hull extract herbal gel (acorn seed coat vaginal gel) with a concentration of 2.5% w/w, the women (patients/candidates) experience more sexual satisfaction, orgasm, vaginal stenosis and a slippery vagina during sexual intercourse when compared to the other groups.

According to one embodiment herein, the sexual satisfaction, orgasm, vaginal stenosis and a slippery vagina during intercourse are found to be less when the Quercus brantii hull extract herbal gel (acorn seed coat vaginal gel) is applied at a dose of in 10 gram and 15 gram dose). After using the herbal gel at a dosage of 20 gram the sexual satisfaction, orgasm, vaginal stenosis and a slippery vagina during intercourse increased.

According to one embodiment herein, 20 g of the herbal gel with Quercus brantii hull extract herbal gel (acorn seed coat vaginal gel) has more effect on the vaginal relaxation syndrome (VRS) and pelvic organ prolapsed (POP).

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A composition for targeting a vaginal relaxation syndrome (VRS) and a pelvic organ prolapsed (POP), the composition comprises:
a herbal extract powder of Quercus brantii seed coat (hull);
a methyl paraben;
an acetic acid;
a de-ionized water; and
a glycerine.

2. The composition according to claim 1, wherein the herbal extract powder of Quercus brantii seed coat (hull) is present in a preset weight % range, and wherein the preset weight % ranges are 1.5% w/w, 2% w/w, 2.5% w/w.

3. The composition according to claim 1, wherein the methyl paraben is present in a preset weight %, and wherein the preset weight % of the methyl paraben is 0.2% w/w.

4. The composition according to claim 1, wherein the herbal gel is administered to women (candidates/participants/volunteers) with a vaginal relaxation syndrome (VRS) and a pelvic organ prolapsed (POP).

5. A method of synthesizing a herbal gel comprising the herbal extract from acorn or oak nut of Quercus brantii for targeting a vaginal relaxation syndrome (VRS) and a pelvic organ prolapsed (POP), the method comprises the steps of :
mixing a herbal extract powder of Quercus brantii seed coat (hull), a methyl paraben, an acetic acid, a de-ionized water and a glycerine; and
obtaining the herbal extract gel of Quercus brantii seed coat (hull) extract.

6. The method according to claim 5, wherein the herbal extract powder of Quercus brantii seed coat (hull) is mixed in a preset weight % range, and wherein the preset weight % ranges are 1.5% w/w, 2% w/w, 2.5% w/w.

7. The method according to claim 5, wherein the methyl paraben is mixed in a preset weight %, and wherein the preset weight % of the methyl paraben is 0.2% w/w.

8. The method according to claim 5, wherein the herbal gel is administered to women (candidates/participants/volunteers).

9. The method according to claim 5, wherein the women (candidates/patients/participants) are administered with plurality of dosage of herbal gel, and wherein the plurality of dosage of herbal gel is 10 gram of herbal gel, 15 gram of herbal gel and 20 gram of herbal gel, and wherein the herbal gel is administered for 3 days.

10. The method according to claim 9, wherein the 10 gram of herbal gel comprises 1.5% w/w Quercus brantii hull/seed coat extract.

11. The method according to claim 9, wherein the 15 gram of herbal gel comprises2% w/w Quercus brantii hull/seed coat extract.

12. The method according to claim 9, wherein the 20 gram of herbal gel comprises and 2.5% w/w Quercus brantii hull/ seed coat extract.

13. The method according to claim 5, wherein the women (patients/volunteers/candidates) experience enhanced sexual satisfaction, an orgasm, a vaginal stenosis and a slippery vagina during the sexual intercourse, after the application of the herbal gel with Quercus brantii hull/seed coat extract in a concentration of 2.5% w/w.

14. The method according to claim 5, wherein 20 gram of the herbal gel with Quercus brantii hull/seed coat extract increases an effect on the women (patients/volunteers/candidates) with a vaginal relaxation syndrome (VRS) and a pelvic organ prolapsed (POP).

15. A method for extracting a herbal extract from acorn or oak nut of Quercus brantii for synthesizing a herbal gel for targeting a vaginal relaxation syndrome (VRS) and a pelvic organ prolapsed (POP), the method comprises the steps of:
collecting the acorn or oak nut from Quercus brantii trees;
drying the acorn or oak nut at a room temperature;
separating wooden shells from the acorn or oak nut seed;
separating seed coat present below the wooden shell from each of the acorn or oak nut;
milling the seed coat into a powder;
mixing the seed coat powder with a solvent in a capped container to get a mixture,
incubating the mixture at a room temperature for a predetermined period of time; and wherein the predetermined time period is 48-72 hours;
agitating the mixture at a regular interval of time for dissolving tannins;
straining the mixture after 48 hours;
pressing the solids of the mixture to extract the solvent and wherein the solvent is methanol extract;
cleaning the solvent by plurality of methods, and wherein the plurality of methods include decanting or filtering;
obtaining herbal extract filtrate by evaporating the methanol extract;
cooling the herbal extract to obtain a powder at a room temperature;
powdering the herbal extract into a powder at a room temperature; and
storing the herbal extract powder in a refrigerator.

16. The method according to claim 15, wherein the solvent is a methanol, and wherein the concentration of the methanol is 80% v/v.
